(19) United States
(12) Patent Application Publication
(10) Pub. No.: US 2014/0287529 A1
(43) Pub. Date: Sep. 25, 2014

(54) METHODS OF NORMALIZING MEASURED DRUG CONCENTRATIONS AND TESTING FOR POTENTIAL NON-COMPLIANCE WITH A DRUG TREATMENT REGIMEN

(71) Applicant: AMERITOX, LTD., BALTIMORE, MD (US)

(72) Inventor: Harry Leider, Baltimore, MD (US)

(73) Assignee: AMERITOX, LTD., BALTIMORE, MD (US)

(21) Appl. No.: 14/216,312

(22) Filed: Mar. 17, 2014

Related U.S. Application Data
(60) Provisional application No. 61/792,472, filed on Mar. 15, 2013.

(51) Int. Cl. G01N 33/94

(52) U.S. Cl. CPC G01N 33/9486
USPC 436/501

(57) ABSTRACT

Methods for monitoring subject compliance with a prescribed treatment regimen are disclosed. In an embodiment, the method comprises measuring a drug level in fluid of a subject and normalizing the measured drug level as a function of one or more parameters associated with the subject. The drug level can be normalized using second order quantile regression. Embodiments of the methods can use both primary and secondary metabolites in the normalization; allow changing variance by dose; allow asymmetry in variance above and below the estimated median values; and/or use analytic variables with stable estimates, such as, for example, variables associated with the percentile for −1 standard deviation, the percentile for 0 standard deviation, and the percentile for +1 standard deviation.

### PRIORITY CLAIM

This application claims priority to U.S. provisional patent application Ser. No. 61/792,472, filed on Mar. 15, 2013, the entire contents of which are incorporated herein by reference and relied upon.

### TECHNICAL FIELD

The present disclosure provides methods for detecting and quantifying a subject's drug use by, inter alia, testing a biological sample from said subject.

### BACKGROUND

Although hydrocodone stands as the most prescribed opioid in the United States, the opioid that is responsible for the most emergency department (ED) visits in the United States is oxycodone. According to the Drug Abuse Warning Network, approximately 77,000 ED visits in 2007 were due to the nonmedical use of oxycodone. The 2007 National Survey on Drug Use and Health estimates that 4.3 million Americans will abuse OXYCONTIN® sometime during the course of their lifetime. Given the propensity for abuse of oxycodone containing medications and high incidence of ED visits associated with abuse, monitoring patients for compliance while being prescribed a pain regimen is an important component of their care.

Because of known dependency risks, subjects on opioid therapy regimens are typically screened periodically to monitor compliance and efficacy of the prescribed therapy. Due to the limits of known screening techniques, however, subjects misusing the prescribed opioid often pass basic screening tests performed at a clinic and continue to receive the opioid. Furthermore, patients treated with opioids for the management of chronic pain also have been documented to under-report their use of medications. As a result, health care professionals often use external sources of information such as interviews with the subject's spouse and/or friends, review of the subject's medical records, input from prescription monitoring programs, and testing of biological samples (e.g., fluids) to detect misuse of drugs and non-compliance with the prescribed opioid regimen.

Known drug screening methods generally can detect the presence or absence of a drug in a sample. Samples of fluids are generally obtained from the subject, for example, urine, blood, or plasma. Such known screening methods do not, however, enable the health care professional reviewing the lab result to determine whether the subject is non-compliant with a prescribed drug regimen.

### SUMMARY

In various embodiments, the present invention provides methods for detecting or monitoring a subject's potential non-compliance with a prescribed drug regimen. In an embodiment, the invention provides a method of identifying a subject at risk of drug misuse. In still other embodiments, the invention provides a method of reducing the risk of drug misuse in a subject by reducing a prescribed daily dose of a drug for the subject or counseling the subject if the drug concentration in fluid of the subject falls outside the confidence intervals or concentration range for the daily dose of the drug. These and other embodiments can comprise performing quantile regression analysis on a normalized drug concentration determined from a fluid sample from a subject.

Embodiments of the invention can identify samples in the lower and upper extremes of each dose distribution. For example, embodiments of the invention can identify samples in the lower 2.5% and the upper 2.5% extremes of the distribution at each dose. Furthermore, relative to known methods, embodiments of the invention can improve differentiation between doses. For example, embodiments of the invention can improve differentiation between doses, relative to methods that use linear regression in which variance adjustment is based on dosage. Still further, embodiments of the invention can use second order quantile regression-based thresholds as standardized residual thresholds. Moreover, embodiments of the invention can obtain a normalized drug concentration from a sample where the raw primary metabolite concentration and/or the raw secondary metabolite concentration are equal to zero.

In another embodiment, the invention uses results of the most likely compliant samples from a normalized database, and the most likely compliant samples exclude samples from subjects identified as high or low metabolizers, subjects with lab abnormalities, subjects with impaired kidney or liver function, subjects using drugs with overlapping metabolites on the same day, and/or subjects taking medication on an inconsistent schedule.

In other embodiments, both primary and secondary metabolites are measured allowing variance changes by dose; allowing asymmetry in variance above and below the estimated median values and/or allowing use of analytic variables with stable estimates, such as, for example, variables associated with the percentile for −1 standard deviation, the percentile for 0 standard deviation, and the percentile for +1 standard deviation.

These embodiments and other embodiments of the invention will be disclosed in further detail herein below.

### BRIEF DESCRIPTION OF THE DRAWINGS

FIG. 1 is a table summarizing drug-specific criteria, quantile regression coefficients, and standard score adjustment coefficients in embodiments of the present disclosure.

FIG. 2 shows: (i) in the upper panel, a quantile regression plot for controlled-release oxycodone (OXYCONTIN®), and (ii) in the lower panel, a final classification plot for OXYCONTIN, according to the present disclosure.

FIG. 3 shows: (i) in the upper panel, a quantile regression plot for oxycodone, and (ii) in the lower panel, a final classification plot for oxycodone, according to the present disclosure.

FIG. 4 shows: (i) in the upper panel, a quantile regression plot for controlled release morphine (MS CONTIN®), and (ii) in the lower panel, a final classification plot for MS CONTIN®, according to the present disclosure.

FIG. 5 shows: (i) in the upper panel, a quantile regression plot for extended release morphine (KADIAN®), and (ii) in the lower panel, a final classification plot for KADIAN®, according to the present disclosure.

FIG. 6 shows: (i) in the upper panel, a quantile regression plot for hydrocodone, and (ii) in the lower panel, a final classification plot for hydrocodone, according to the present disclosure.

FIG. 7 shows: (i) in the upper panel, a quantile regression plot for a combination of OXYCONTIN® and controlled-release oxycodone, and (ii) in the lower panel, a final classification plot for the combination of OXYCONTIN® and controlled-release oxycodone, according to the present disclosure.

FIG. 8 shows: (i) in the upper panel, a quantile regression plot for methadone, and (ii) in the lower panel, a final classification plot for methadone, according to the present disclosure.

FIG. 9 shows: (i) in the upper panel, a quantile regression plot for normalized OXYCONTIN® concentrations obtained from Equation 1 according to the present disclosure, and (ii) in the lower panel, a quantile regression plot for normalized OXYCONTIN® concentrations obtained from a normalization equation based solely on raw OXYCONTIN® concentration, lean body weight and creatinine level.

FIG. 10 shows: (i) in the upper panel, a quantile regression plot for normalized oxycodone concentrations obtained from Equation 1 according to the present disclosure, and (ii) in the lower panel, a quantile regression plot for normalized oxycodone concentrations obtained from a normalization equation based solely on raw oxycodone concentration, lean body weight and creatinine level.

FIG. 11 shows: (i) in the upper panel, a quantile regression plot for normalized MS CONTIN® concentrations obtained from Equation 1 according to the present disclosure, and (ii) in the lower panel, a quantile regression plot for normalized MS CONTIN® concentrations obtained from a normalization equation based solely on raw MS CONTIN® concentration, lean body weight and creatinine level.

FIG. 12 shows: (i) in the upper panel, a quantile regression plot for normalized extended release morphine (KADIAN®) concentrations obtained from Equation 1 according to the present disclosure, and (ii) in the lower panel, a quantile regression plot for normalized KADIAN® concentrations obtained from a normalization equation based solely on raw KADIAN® concentration, lean body weight and creatinine level.

FIG. 13 shows: (i) in the upper panel, a quantile regression plot for normalized hydrocodone concentrations obtained from Equation 1 according to the present disclosure, and (ii) in the lower panel, a quantile regression plot for normalized hydrocodone concentrations obtained from a normalization equation based solely on raw hydrocodone concentration, lean body weight and creatinine level.

FIG. 14 shows: (i) in the upper panel, a quantile regression plot for normalized concentrations of a combination of OXYCONTIN® and controlled-release oxycodone obtained from Equation 1 according to the present disclosure, and (ii) in the lower panel, a quantile regression plot for normalized concentrations of OXYCONTIN® and controlled release oxycodone obtained from a normalization equation based solely on raw concentrations of OXYCONTIN® and controlled release oxycodone, lean body weight and creatinine level.

FIG. 15 shows: (i) in the upper panel, a quantile regression plot for normalized methadone concentrations obtained from Equation 1 according to the present disclosure, and (ii) in the lower panel, a quantile regression plot for normalized methadone concentrations obtained from a normalization equation based solely on raw methadone concentration, lean body weight and creatinine level.

FIG. 16 displays and compares results obtained from quantile regression analysis of normalized drug concentrations obtained from Equation 1 in the upper panel and a quantile regression analysis of normalized drug concentrations obtained from a normalization equation based solely on raw drug concentration, lean body weight and creatinine level.

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

As used herein, the singular form of a word includes the plural, and vice versa, unless the context clearly dictates otherwise. Thus, the references “a”, “an”, and “the” are generally inclusive of the plurals of the respective terms. For example, reference to “an embodiment” or “a method” includes a plurality of such “embodiments” or “methods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. The terms “comprising” or “including” are intended to include embodiments encompassed by the terms “consisting essentially of” and “consisting of.” Similarly, the term “consisting essentially of” is intended to include embodiments encompassed by the term “consisting of”.

### Therapeutic Regimens

In one embodiment, the present invention provides a method of detecting non-compliance or potential non-compliance with a prescribed drug regimen in a subject. The term “non-compliance” as used herein refers to any substantial deviation from a course of treatment that has been prescribed by a physician, nurse, nurse practitioner, physician's assistant, or other health care professional. A substantial deviation from a course of treatment may include any intentional or unintentional behavior by the subject that increases or decreases the amount, timing or frequency of drug ingested compared to the prescribed therapy.

Non-limiting examples of substantial deviations from a course of treatment include: taking more of the drug than prescribed, taking less of the drug than prescribed, taking the drug more often than prescribed, taking the drug less often than prescribed, intentionally diverting at least a portion of the prescribed drug, unintentionally diverting at least a portion of the prescribed drug, etc. For example, a subject substantially deviates from a course of treatment by taking about 5% to about 1000% of the prescribed daily dose or prescribed drug regimen, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 105%, about 110%, about 115%, about 120%, about 125%, about 150%, about 175%, about 200%, about 225%, about 250%, about 275%, about 300%, about 350%, about 400%, about 450%, about 500%, about 550%, about 600%, about 650%, about 700%, about 750%, about 800%, about 850%, about 900%, about 950%, or about 1000% of the prescribed drug regimen.

A subject may also substantially deviate from a course of treatment by taking about 5% to about 1000% more or less than the prescribed dose, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 225%, about 250%, about 275%, about 300%, about 350%, about 400%, about 450%, about 500%, about 550%, about 600%, about 650%, about 700%, about 750%, about 800%, about 850%, about 900%, about 950%, or about 1000% less than the prescribed dose. A subject may also substantially deviate from a course of treatment by, for example, taking the prescribed dose of a drug about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 225%, about 250%, about 275%, about 300%, about 350%, about 400%, about 450%, about 500%, about 550%, about 600%, about 650%, about 700%, about 750%, about 800%, about 850%, about 900%, about 950%, or about 1000% more often or less often than specified in the course of treatment or prescribed in the drug regimen.

In another embodiment, a subject according to the present invention is prescribed a daily dose of a drug. The term “daily dose” or “prescribed daily dose” as used herein refers to any periodic administration of a drug to the subject over a given period of time, for example per hour, per day, per every other day, per week, per month, per year, etc. Preferably the daily dose or prescribed daily dose is the amount of the drug prescribed to a subject in any 24-hour period. The drug may be administered according to any method known in the art including, for example, orally, intravenously, topically, transdermally, subcutaneously, rectally, etc. The prescribed daily dose of the drug may be approved by the Food & Drug Administration (“FDA”) for a given indication. In the alternative, a daily dose or a prescribed daily dose may be an unapproved or “off-label” use for a drug for which FDA has approved other indications. As a non-limiting example, FDA has approved oxycodone HCI controlled-release tablets (OXYCONTIN®) for use in the management of moderate to severe pain in 10 mg, 15 mg, 20 mg, 30 mg, 40 mg, 60 mg, 80 mg, 160 mg tablets. Any use of oxycodone HCI controlled-release tablets (OXYCONTIN®) other than to manage moderate to severe pain or at other than approved doses is an “off-label” use.

In various embodiments, methods according to the present invention involve the step of determining a prescribed dose of a drug. The term “determining a prescribed dose” as used herein refers to any method known to those in the art to ascertain, discover, deduce, or otherwise learn the dose of a particular drug that has been prescribed to the subject. Non-limiting examples include subject interview, consultation with the subject's medical history, consultation with another health care professional familiar with the subject, consultation with a medical record associated with the subject, etc.

The term “drug” as used herein refers to an active pharmaceutical ingredient (“API”) and its metabolites, decomposition products, enantiomers, diastereomers, derivatives, etc.

In an embodiment, the drug is an opioid. The term “opioid” as used herein refers to any natural, endogenous, synthetic, or semi-synthetic compound that binds to opioid receptors. Non-limiting examples of opioids include: codeine, morphine, thebaine, oripavine, diacetylmorphine, dihydrocodeine, hydrocodone, hydromorphone, nicomorphone, oxycodone, oxymorphone, fentanyl, alphamethylfentanyl, alfentanil, sufentanil, remifentanil, carfentanyl, ohmefentanyl, pethidine, keobemidone, desmethylprodine, (“MPPP”), allylprodine, prodine, 4-phenyl-1-(2-phenylethyl)piperidin-4-yl acetate (“PEPAP”), propoxyphene, dextropropoxyphene, dextromoramide, bezitramide, piritramide, methadone, dipipanone, levomathadyl acetate (“LAAM”), difenoxin, diphenoxylate, loperamide, dezocine, pentazocine, phenazocine, buprenorphine, dihydroetorphine, etorphine, butorphanol, nalbuphine, levorphanol, levomethorphan, lefetamine, meptazinol, tilidine, tramadol, tapentadol, nalmefene, naloxone, naltrexone, methadone, oxazepam, lorazepam, alprazolam, diazepam, derivatives thereof, metabolites thereof, prodrugs thereof, controlled-release formulations thereof, extended-release formulations thereof, sustained-release formulations thereof, and combinations of the foregoing.

In an embodiment, a method according to the present invention confirms a subject's non-adherence to a chronic opioid therapy (“COT”). The term “chronic opioid therapy” as used herein refers to any short-term, mid-term, or long-term treatment regimen comprising at least one opioid. As a non-limiting example, a subject suffering chronic pain may ingest a daily dose of oxycodone to relieve persistent pain resulting from trauma, chronic conditions, etc. COT is generally prescribed to a subject in need of such therapy; subjects on COT are typically monitored periodically by a health care professional for addiction, tolerance, or other common outcomes associated with COT. In one embodiment, a method according to the present invention assists a health care professional in confirming a subject's adherence or non-adherence to a COT regimen.

Subjects on COT sometimes develop an addiction to the prescribed opioid. Studies have shown that a subject on COT is more likely to develop an addiction to a prescribed opioid when he or she has a history of aberrant drug-related behavior, or is at high risk of aberrant drug-related behavior. The term “aberrant drug-related behavior” as used herein refers to any behavioral, genetic, social, or other characteristic of the subject that tends to predispose the subject to development of an addiction for an opioid.

Non-limiting examples of such risk factors include a history of drug abuse, a history of opioid abuse, a history of non-opioid drug abuse, a history of alcohol abuse, a history of substance abuse, a history of prescription drug abuse, a low tolerance to pain, a high rate of opioid metabolism, a history of purposeful over-sedation, negative mood changes, intoxicated appearance, an increased frequency of appearing unkempt or impaired, a history of auto or other accidents, frequent early renewals of prescription medications, a history of or attempts to increasing dose without authorization, reports of lost or stolen medications, a history of contemporaneously obtaining prescriptions from more than one doctor, a history of altering the route of administering drugs, a history of using pain relief medications in response to stressful situations, insistence on certain medications, a history of contact with street drug culture, a history of alcohol abuse, a history of illicit drug abuse, a history of hoarding or stockpiling medications, a history of police arrest, instances of abuse or violence, a history of visiting health care professionals without an appointment, a history of consuming medications in excess of the prescribed dose, multiple drug allergies and/or intolerances, frequent office calls and visits, a genetic mutation that up-regulates or down-regulates production of drug metabolizing enzymes, a reduced-function CYP2D6 allele, and/or a non-functional CYP2D6 allele.

In another embodiment, the drug is a benzodiazepine, a stimulant, or any medication that is chronically administered. In one embodiment, the drug is an antipsychotic drug. The term “antipsychotic drug” as used herein refers to any natural, endogenous, synthetic, or semi-synthetic compound that manages and/or treats psychosis; binds to dopamine receptors, glutamate receptors and/or serotonin receptors as an agonist and/or an antagonist; and/or affects the dopamine pathway, the glutamate pathway and/or the serotonin pathway. Non-limiting examples of antipsychotic drugs include: amisulpride, aripiprazole, asenapine, azaperone, benperidol, bifeprunox, blonanserin, clotiapine, clopenthixol, chlorpromazine, chlorprothixene, clozapine, cyamemazine, droperidol, flupentixol, fluphenazine, haloperidol, iloperidone, lenperone, levomepromazine, loxapine, lurasidone, melperone, mesoridazine, methotrimeprazine, molindone, mosapramine, olanzapine, paliperidone, periciazine, perospirone, perphenazine, pimavanserin, pimozide, prochlorperazine, promazine, promethazine, quetiapine, remoxipride, risperidone, sertindole, sulpiride, tetrabenazine, thioridazine, thiothixene, trifluoperazine, triflupromazine, triperidol, vabicaserin, ziprasidone, zotepine, zuclopenthixol, derivatives thereof, metabolites thereof, prodrugs thereof, controlled-release formulations thereof, extended-release formulations thereof, sustained-release formulations thereof, and combinations of the foregoing.

In an embodiment, the present invention assists a health care professional in assessing a risk that a subject is misusing a prescribed drug. For example, based on the determinations obtained by the quantile regression analysis performed in embodiments of the present invention, a healthcare worker can intervene (e.g. via counseling, modifying the subject's regiment/dose, etc.) in the subject's misuse on the basis of the risk assessment.

### Sample Measurement

Methods according to the present invention may be used to determine the amount of a wide variety of drugs in fluids of a subject. When the fluid analyzed is urine, for example, methods according to the present invention may be used to determine the amount of any drug that can be measured in a urine sample.

In an embodiment, the amount of a drug in a subject is determined by analyzing a fluid of the subject. The term “fluid” as used herein refers to any liquid or pseudo-liquid obtained from the subject. Non-limiting examples include urine, blood, plasma, saliva, mucus, and the like. In an embodiment, the fluid is urine.

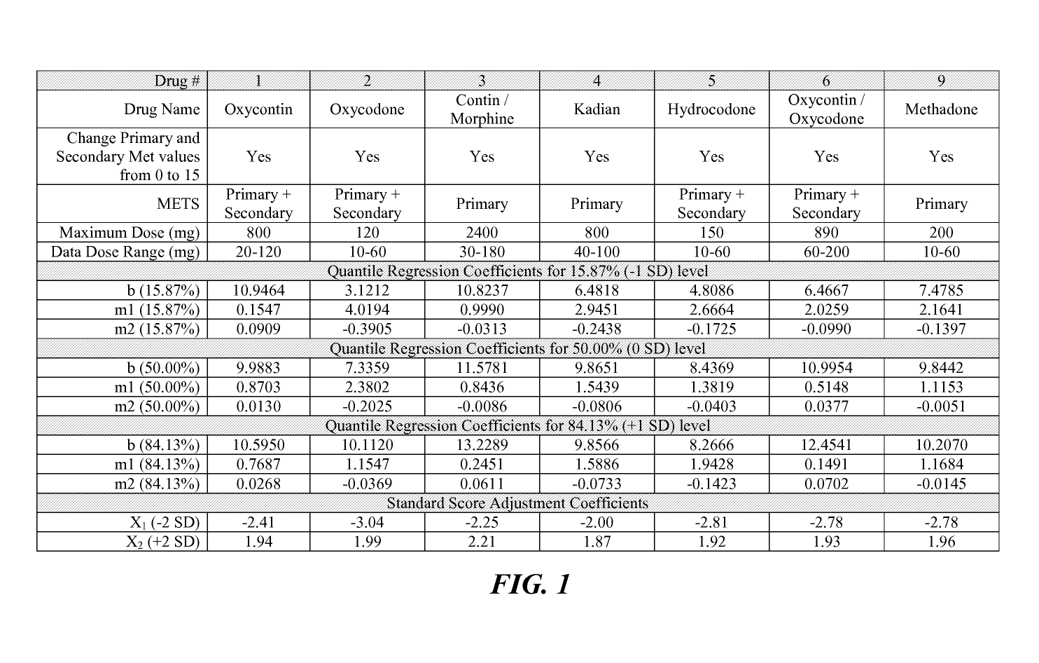

In an embodiment, the prescribed daily dose of the drug is compared to a maximum value before proceeding with the method. For example, an embodiment includes comparing the prescribed daily dose to a maximum value before determining the amount of the drug in the subject. In a related embodiment, the amount of the drug is only determined if the prescribed daily dose is equal to or less than the maximum value. As shown in the table in FIG. 1, non-limiting examples of maximum values include 800 mg/day of controlled-release oxycodone (OXYCONTIN®), 120 mg/day oxycodone, 2400 mg/day controlled release morphine (MS CONTIN®) or morphine, 800 mg/day extended release morphine (KADIAN®), 150 mg/day hydrocodone, 890 mg/day total of the combination of controlled-release oxycodone (OXYCONTIN®) and oxycodone, and 200 mg/day methadone.

Determining the amount of a drug in fluid of the subject may be accomplished by use of any method known to those skilled in the art. Non-limiting examples for determining the amount of a drug in fluid of a subject include fluorescence polarization immunoassay (“FPIA,” Abbott Diagnostics), mass spectrometry (MS), gas chromatography-mass spectrometry (GC-MS-MS), liquid chromatography-mass spectrometry (LC-MS-MS), and the like. In one embodiment, LC-MS-MS methods known to those skilled in the art are used to determine a raw level, amount or concentration of a drug in urine of the subject. In one embodiment, a raw level or concentration of a drug in fluid of a subject is measured and reported as a ratio, percent, or in relationship to the amount of fluid. The amount of fluid may be expressed as a unit volume, for example, in L, mL, μL, pL, ounce, etc. In one embodiment, the raw amount of a drug in fluid of a subject may be expressed as an absolute level or value, for example, in g, mg, μg, ng, pg, etc.

In an embodiment, the level, concentration or amount of a drug determined in fluid of a subject is normalized. The term “normalized” as used herein refers to a level or concentration of a drug that has been adjusted to correct for one or more parameters associated with the subject. Non-limiting examples of parameters include: sample fluid pH, sample fluid specific gravity, sample fluid creatinine concentration, subject height, subject weight, subject age, subject body mass index, subject gender, subject lean body mass, and subject body surface area. Parameters may be measured by any means known in the art. For example, sample fluid pH may be measured using a pH meter, litmus paper, test strips, etc.

In an embodiment, the normalized drug concentration is determined using parameters comprising subject age, subject weight, subject gender and sample fluid creatinine concentration. In a related embodiment, the normalized drug concentration is determined without using sample fluid pH or subject lean body mass. In another related embodiment, the normalized drug concentration is determined from the primary metabolite concentration using parameters consisting of subject age, subject weight, subject gender and sample fluid creatinine concentration. In yet another related embodiment, the normalized drug concentration is determined from the primary metabolite concentration and the secondary metabolite concentration using parameters consisting of primary metabolite concentration, secondary metabolite concentration, subject age, subject weight, subject gender and sample fluid creatinine concentration. The primary metabolite can be the opioid itself.

In an embodiment, the raw drug concentration measured in fluid of the subject is normalized as a function of subject age, subject weight, subject gender and sample fluid creatinine concentration according to the following normalization equation (hereafter “Equation 1”):

$$ADJUSTED\_MET = ln ( \frac{METS * NEW\_AGE * WEIGHT ( lbs ) * NEW\_MF}{UR\_CREAT} ) \qquad ( 1 )$$

where In is the natural log, ADJUSTED_MET is the normalized drug level; METS is the total raw concentration in ng/mL of the primary metabolite and optionally the secondary metabolite; NEW_AGE is (140—subject age in years); WEIGHT is the weight of the subject in pounds; NEW_MF is the gender correction factor, 0.85 if the subject is female, 1.0 if the subject is male; and UR_CREAT is the urine creatinine value in mg/dL. If both the primary metabolite concentration and the secondary metabolite concentration are used in Equation 1, METS is the total of the raw concentration in ng/mL of the primary metabolite added to the raw concentration in ng/mL of the secondary metabolite.

In an embodiment, if the primary metabolite concentration is measured as zero, the primary metabolite concentration is used in Equation 1 as a different value, such as, for example, a predetermined minimum primary metabolite value for use in Equation 1. Additionally or alternatively, if the secondary metabolite concentration is measured as zero, the secondary metabolite concentration is used in Equation 1 as a different value, such as, for example, a predetermined minimum secondary metabolite value for use in Equation 1. As a non-limiting example, the predetermined minimum primary metabolite value and/or the predetermined minimum secondary metabolite value for use in Equation 1 can be 15 ng/mL.

In a related embodiment, for a subject prescribed controlled-release oxycodone (OXYCONTIN®), a normalized drug level is determined from a raw level of the primary metabolite and the secondary metabolite as a function of subject age, subject weight, subject gender and sample fluid creatinine concentration, according to Equation 1. In a related embodiment, controlled-release oxycodone (OXYCONTIN®) is the only opioid prescribed to the subject.

In another related embodiment, for a subject prescribed oxycodone, a normalized drug level is determined from a raw level of the primary metabolite and the secondary metabolite as a function of subject age, subject weight, subject gender and sample fluid creatinine concentration, according to Equation 1. In a related embodiment, oxycodone is the only opioid prescribed to the subject.

In another related embodiment, for a subject prescribed controlled-release morphine (MS CONTIN®) or morphine, a normalized drug level is determined from a raw level of the primary metabolite as a function of subject age, subject weight, subject gender and sample fluid creatinine concentration, according to Equation 1. In a related embodiment, controlled-release morphine (MS CONTIN®) or morphine is the only opioid prescribed to the subject.

In another related embodiment, for a subject prescribed extended release morphine (KADIAN®), a normalized drug level is determined from a raw level of the primary metabolite as a function of subject age, subject weight, subject gender and sample fluid creatinine concentration, according to Equation 1. In a related embodiment, extended release morphine (KADIAN®) is the only opioid prescribed to the subject.

In another related embodiment, for a subject prescribed hydrocodone, a normalized drug level is determined from a raw level of the primary metabolite and the secondary metabolite as a function of subject age, subject weight, subject gender and sample fluid creatinine concentration, according to Equation 1. In a related embodiment, hydrocodone is the only opioid prescribed to the subject.

In another related embodiment, for a subject prescribed both controlled-release oxycodone (OXYCONTIN®) and oxycodone, a normalized drug level is determined from a raw level of the primary metabolite and the secondary metabolite as a function of subject age, subject weight, subject gender and sample fluid creatinine concentration, according to Equation 1. In a related embodiment, controlled-release oxycodone (OXYCONTIN®) and oxycodone are the only opioids prescribed to the subject.

In another related embodiment, for a subject prescribed methadone, a normalized drug level is determined from a raw level of the primary metabolite 2-ethylidene-1,5-dimethyl-3,3-diphenylpyrrolidine (EDDP) as a function of subject age, subject weight, subject gender and sample fluid creatinine concentration, according to Equation 1. In a related embodiment, methadone is the only opioid prescribed to the subject.

In an embodiment, the concentration or level of drug in fluid of the subject is a steady state concentration or level. The term “steady state” as used herein refers to an equilibrium level or concentration of a drug obtained at the end of a certain number of administrations (e.g. 1 to about 5). Steady state is achieved when the concentration or level of the drug will remain substantially constant if the dose and the frequency of administrations remain substantially constant.

The new normalization equation was created as follows. At steady state, the time averaged plasma concentration of drug D is given by the following Equation 2:

$${C}_{p}^{D} = \frac{D * F}{τ * {CL}^{D}} \qquad ( 2 )$$

where Cₚ^{D}=steady state concentration of drug D in plasma, D=drug dose, F=fraction of drug absorbed from the gut to the plasma, τ=drug dosing period, and CL^{D}=clearance of drug D.

Therefore, rearranging Equation (2) results in:

$$\frac{D}{τ} = \frac{{C}_{p}^{D} * {CL}^{D}}{F} \qquad ( 3 )$$

The standard equation for urine clearance of drug D is given by:

$${CL}^{D} = \frac{{C}_{u}^{D} * {Q}_{U}}{{C}_{p}^{D}} \qquad ( 4 )$$

where Q_{U}=volume flow of urine and Cᵤ^{D}=concentraton of drug D in urine.

Substituting Equation (4) into Equation (3) yields:

$$\frac{D}{τ} = \frac{{C}_{p}^{D} * {C}_{u}^{D} * {Q}_{U}}{{C}_{p}^{D} * F} = \frac{{C}_{u}^{D} * {Q}_{U}}{F} \qquad ( 5 )$$

In a similar manner to equation (4), the clearance of creatinine (CR) is defined as:

$${CL}^{CR} = \frac{{C}_{u}^{CR} * {Q}_{U}}{{C}_{p}^{CR}} \qquad ( 6 )$$

where Cᵤ^{CR} and Cₚ^{CR}=concentraton of creatinine in urine and plasma respectively

The Cockroft-Gault formula for creatinine clearance is given by:

$${CL}^{CR} = \frac{( 140 - age ) * ( weight ) * ( .85 * f + m )}{72 * {C}_{p}^{CR}} \qquad ( 7 )$$

where weight is in kg, Cₚ^{CR} is in mg/dl, and f=1 and m=0 for female, and f=0 and m=1 for male.

From Equation (65), solving for Q_{U} results in the following equation:

$${Q}_{U} = \frac{{CL}^{CR} * {C}_{p}^{CR}}{{C}_{u}^{CR}} \qquad ( 8 )$$

Substituting Equation (7) into Equation (8) yields:

$${Q}_{U} = \frac{{C}_{p}^{CR} * ( 140 - age ) * weight * ( .85 * f + m )}{72 * {C}_{p}^{CR} * {C}_{u}^{CR}}$$

or:

$${Q}_{U} = \frac{( 140 - age ) * weight * ( .85 * f + m )}{72 * {C}_{u}^{CR}} \qquad ( 9 )$$

Substituting (9) into (5) yields:

$$\frac{D}{τ} = \frac{{C}_{u}^{D} * ( 140 - age ) * weight * ( .85 * f + m )}{72 * {C}_{u}^{CR} * F} \qquad ( 10 )$$

The rate of reabsorption of the drug by the kidney is a function of the relative ionic concentration (a higher ionic concentration results in lower reabsorption and an effectively higher urine concentration of the drug). Therefore, the following calculations adjust for this factor.

From the Henderson-Hasselbach equation:

$${pH}_{u} = {pK}_{a} + {log}_{10} ( \frac{[ {A}^{-} ]}{[ HA ]} ) \qquad ( 11 )$$

where pHᵤ is the urine pH, [A⁻] and [HA] are the drug anion and drug acid concentrations respectively.

Then rearranging Equation (11) for a drug that is an acid yields:

$${I}_{f} = {k}_{pHacid} {10}^{( {pH}_{u} - {pK}_{a} )} = {k}_{pHacid} \frac{[ {A}^{-} ]}{[ HA ]} \qquad ( 12 )$$

where k_{pHacid} is proportionality constant relating anion/acid concentration ratio to change in urinary excretion related to urine pHᵤ change, pKₐ is the acid dissociation constant for the drug and I_{f} is the ion factor impacting the dose to urine level.

Or in like manner, for a drug that is a base, rearranging Equation (11) for a drug that is an acid yields:

$${I}_{f} = {k}_{pHbase} {10}^{( 14 - {pH}_{u} - {pK}_{a} )} = {k}_{pHbase} \frac{[ {BH}^{+} ]}{[ B ]} \qquad ( 13 )$$

Then substituting Equation (12) or Equation (13) into Equation (9) yields:

$$\frac{D}{τ} = \frac{{C}_{u}^{D} * ( 140 - age ) * weight * ( .85 * f + m )}{72 * {C}_{u}^{CR} * F * {I}_{f}} \qquad ( 14 )$$

or simplifying Equation (14) by combining the constants for a drug that is a acid yields:

$$\frac{D}{τ} = {K}_{Da} * \frac{{C}_{u}^{D} * ( 140 - age ) * weight * ( .85 * f + m )}{{C}_{u}^{CR} * {10}^{( {pH}_{u} )}} \frac{D}{τ} = {K}_{Da} * \frac{{C}_{u}^{D} * ( 140 - age ) * weight * ( .85 * f + m )}{{C}_{u}^{CR} * {10}^{( {pH}_{u} )}} \qquad ( 15 )$$

where the constant

$${K}_{Da} = \frac{{10}^{( {pK}_{a} )}}{72 * F * {k}_{pHacid}}$$

For a drug that is a base:

$$\frac{D}{τ} = {K}_{Db} * \frac{{C}_{u}^{D} * ( 140 - age ) * weight * ( .85 * f + m )}{{C}_{u}^{CR} * {10}^{( - {pH}_{u} )}} \qquad ( 15 )$$

where the constant

$${K}_{Db} = \frac{{10}^{( {pK}_{a} - 14 )}}{72 * F * {k}_{pHbase}} .$$

In an embodiment, the normalized drug level obtained from Equation 1 can be used in subsequent steps of the method, if any.

### Determining Quantile Regression (QR) Dose-Based Values

In an embodiment, quantile regression (QR) dose-based values are determined. A QR dose-based value can be determined for each of a plurality of standard deviation values, and the standard deviation values are relative to a mean normalized drug concentration derived from samples of a population of subjects. For example, the QR dose-based values can be determined for the −1 standard deviation, the 0 standard deviation, and the +1 standard deviation from the mean normalized drug concentration derived from the population of subjects. As a non-limiting example, the −1 standard deviation, the 0 standard deviation, and the +1 standard deviation can correspond to the 15.87% percentile, the 50% percentile and the 84.13% percentile of the population of subjects, respectively. The term “a population” as used herein refers to any group or selection of subjects. In a related embodiment, the mean normalized drug concentration derived from the samples of the population of subjects is provided by a normalized database.

In a related embodiment, one or a plurality of subjects are assigned to a population. As used herein a “plurality of subjects” refers to two or more subjects, for example about 2 subjects, about 3 subjects, about 4 subjects, about 5 subjects, about 6 subjects, about 7 subjects, about 8 subjects, about 9 subjects, about 10 subjects, about 15 subjects, about 20 subjects, about 25 subjects, about 30 subjects, about 35 subjects, about 40 subjects, about 45 subjects, about 50 subjects, about 55 subjects, about 60 subjects, about 65 subjects, about 70 subjects, about 75 subjects, about 80 subjects, about 85 subjects, about 90 subjects, about 95 subjects, about 100 subjects, about 110 subjects, about 120 subjects, about 130 subjects, about 140 subjects, about 150 subjects, about 160 subjects, about 170 subjects, about 180 subjects, about 190 subjects, about 200 subjects, about 225 subjects, about 250 subjects, about 275 subjects, about 300 subjects, about 325 subjects, about 350 subjects, about 375 subjects, about 400 subjects, about 425 subjects, about 450 subjects, about 475 subjects, about 500 subjects, about 525 subjects, about 550 subjects, about 575 subjects, about 600 subjects, about 625 subjects, about 650 subjects, about 675 subjects, about 700 subjects, about 725 subjects, about 750 subjects, about 775 subjects, about 800 subjects, about 825 subjects, about 850 subjects, about 875 subjects, about 900 subjects, about 925 subjects, about 950 subjects, about 975 subjects, about 1000 subjects, about 1250 subjects, about 1500 subjects, about 1750 subjects, about 2000 subjects, about 2250 subjects, about 2500 subjects, about 2750 subjects, about 3000 subjects, about 3500 subjects, about 4000 subjects, about 4500 subjects, about 5000 subjects, about 5500 subjects, about 6000 subjects, about 6500 subjects, about 7000 subjects, about 7500 subjects, about 8000 subjects, about 8500 subjects, about 9000 subjects, about 9500 subjects, or about 10000 subjects. As used herein with respect to a population, the term “subject” is synonymous with the term “member” and refers to an individual that has been assigned to the population. In one embodiment, subpopulations may be established for a plurality of daily doses of a drug.

In an embodiment, a plurality of subjects of a population are each prescribed the same daily dose of a drug. In another embodiment, a plurality of subjects assigned to one subpopulation are each prescribed a first daily dose of a drug while a plurality of subjects assigned to a second, different subpopulation are each prescribed a second, different daily dose of a drug. In an embodiment, a plurality of subjects assigned to a population or subpopulation are each prescribed a daily dose of a drug for a time sufficient to achieve steady state. The term “time sufficient to achieve steady state” refers to the amount of time required, given the pharmacokinetics of the particular drug and the dose administered to the subject, to establish a substantially constant concentration or level of the drug assuming the dose and the frequency of administrations remain substantially constant. The time sufficient to achieve steady state may be determined from literature or other information corresponding to the drug. For example, labels or package inserts for FDA approved drugs often include information regarding typical times sufficient to achieve steady state plasma concentrations from initial dosing. Other non-limiting means to determine the time sufficient to achieve steady state include experiment, laboratory studies, analogy to similar drugs with similar absorption and excretion characteristics, etc.

Assignment of subjects to a population or subpopulation may be accomplished by any method known to those skilled in the art. For example, subjects may be assigned randomly to one of a plurality of subpopulations. In an embodiment, subjects are screened for one or more parameters before or after being assigned to a population. For example, subjects featuring one or more parameters that may tend to affect fluid levels of a drug may be excluded from a population, may not be assigned to a population, may be assigned to one of a plurality of subpopulations, or may be removed from a population or subpopulation during or after a data collection phase of a study. Subjects may be excluded from a population based on the presence or absence of one or more exclusion criteria such as high opioid metabolism, low opioid metabolism, lab abnormalities, impaired kidney or liver function, use of drugs with overlapping metabolites on the same day, or an inconsistent schedule of medication administration, as non-limiting examples.

In an embodiment, the mean normalized drug concentration is derived from samples of a most likely compliant population of subjects. In such an embodiment, the QR dose-based values are determined for the plurality of standard deviation values from this mean normalized drug concentration. For example, the QR dose-based values can be determined for the −1 standard deviation, the 0 standard deviation, and the +1 standard deviation from the mean normalized drug concentration of the most likely compliant population of subjects. In a related embodiment, the mean normalized drug concentration derived from the samples of the most likely compliant population of subjects is provided by a normalized database. In an embodiment, the most likely compliant population of subjects omits subjects identified as high or low metabolizers, subjects with lab abnormalities, subjects with impaired kidney or liver function, subjects using drugs with overlapping metabolites on the same day, and subjects taking medication on an inconsistent schedule.

In an embodiment, the QR dose-based values are determined using the natural log of the dose in combination with drug and dose-based coefficients, such as, for example, the drug and dose-based coefficients in the table in FIG. 1. The drug and dose-based coefficients can be derived from a population of subjects, such as, for example, a population of most likely compliant subjects as discussed above. Alternatively, the drug and dose-based coefficients can be derived from a population without regard to the likelihood of compliance of the population. Preferably, the drug and dose-based coefficients are derived from the same population from which the mean normalized drug concentration is derived.

As a non-limiting example, the table in FIG. 1 has the drug and dose-based coefficients associated with the −1 standard deviation, the 0 standard deviation, and the +1 standard deviation, which in this example are the 15.87% percentile, the 50% percentile and the 84.13% percentile, respectively. The following general equation can be used to determine the QR dose-based value for a specific percentile:

QRᵢ=bᵢ+(m1ᵢ*LN_DOSE)+(m2ᵢ*LN_DOSE²)

where I=percentile and LN_DOSE=the natural log of the prescribed daily dose.

Using the drug and dose-based coefficients from the table in FIG. 1, the QR dose-based value equation can be employed for the 15.87% percentile, the 50% percentile and the 84.13% percentile as follows:

QR_{15.87}=10.9464+(0.1547*LN_DOSE)+(0.0909*LN_DOSE²)

QR_{50.00}=9.9883+(0.8703*LN_DOSE)+(0.0130 LN_DOSE²)

QR_{84.13}=10.5950+(0.7687*LN_DOSE)+(0.0268*LN_DOSE²)

In an embodiment, the QR dose-based values obtained from the QR dose-based value equation can be used in subsequent steps of the method, if any.

### Determining a Preliminary Standard Score

In an embodiment, the QR dose-based values can be used to determine a preliminary standard score for the normalized drug concentration of the subject. The preliminary standard score can be determined by comparing the normalized drug concentration obtained by Equation 1, ADJUSTED_{MET}, to the QR dose-based value for the 50% percentile as follows:

If ADJUSTED_{MET}=QR_{50.00}, then

SS_{preliminary}=0.00

If ADJUSTED_{MET}>QR_{50.00}, then

$${SS}_{Preliminary} = \frac{( {ADJUSTED}_{MET} - {QR}_{50.00} )}{( {QR}_{84.13} - {QR}_{50.00} )}$$

If ADJUSTED_{MET}<QR_{50.00} then

$${SS}_{Preliminary} = \frac{- ( {QR}_{50.00} - {ADJUSTED}_{MET} )}{( {QR}_{50.00} - {QR}_{15.87} )}$$

In an embodiment, the preliminary standard score can be used in subsequent steps of the method, if any.

### Determining a Final Standard Score

In an embodiment, the preliminary standard score is used to obtain a final standard score. In a related embodiment, the preliminary standard score and final standard score adjustment variables are used to obtain the final standard score, and the final standard score adjustment variables can be determined using adjustment coefficients, such as, for example, the adjustment coefficients X₁ and X₂ in the table in FIG. 1. The final standard score adjustment variables can be determined using the adjustment coefficients X₁ and X₂ as follows:

$${m}_{ss} = \frac{4.00}{{X}_{2} - {X}_{1}}$$

$${b}_{ss} = - 2.00 - ( {m}_{ss} * {X}_{1} )$$

The final standard score can be determined as follows:

SS_{Final}=(m_{SS}*SS_{Preliminary})+b_{SS}

In an embodiment, the final standard score can be used in subsequent steps of the method, if any.

### Determining Potential Compliance or Non-Compliance

In an embodiment, a subject's potential non-compliance with a prescribed treatment protocol or treatment regimen is assessed or analyzed by comparing the subject's final standard score to a lower threshold and an upper threshold. In a related embodiment, the lower threshold and the upper threshold can be predetermined standardized values that are applicable to all samples and all opioids to which the method is applied. In a related embodiment, the subject is non-compliant if the subject's final standard score is less than the lower threshold or greater than the upper threshold, and the subject is compliant if the subject's final standard score is greater than or equal the lower threshold and less than or equal to the upper threshold. As a non-limiting example, the lower threshold can be −2.0 and the upper threshold can be +2.0, and the subject is non-compliant if the subject's final standard score is less than −2.0 or greater than +2.0 and is compliant if the subject's final standard score is greater than or equal to −2.0 and less than or equal to +2.0.

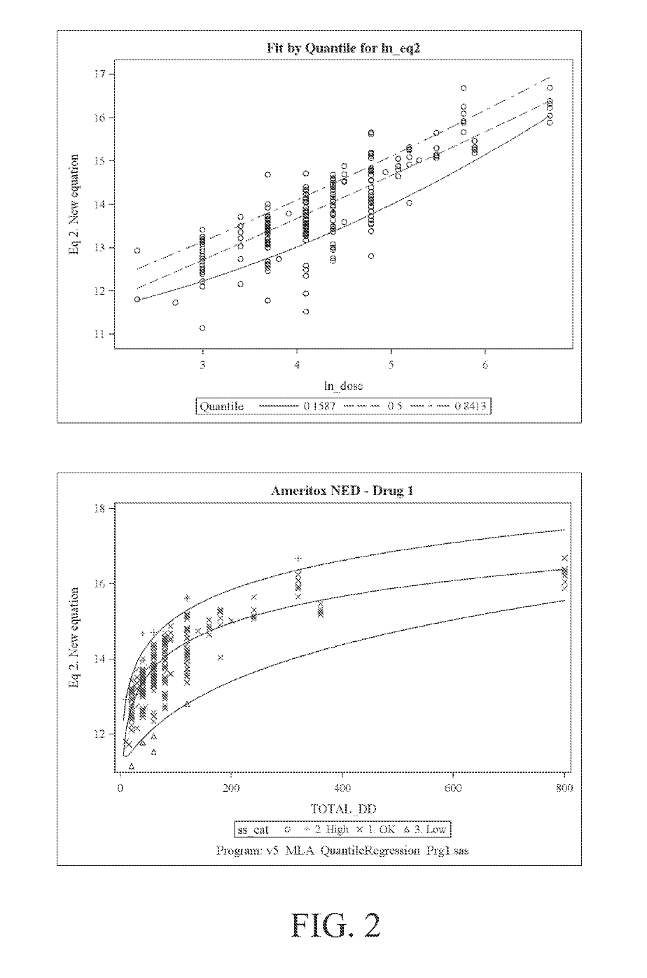

For controlled-release oxycodone (OXYCONTIN®), the upper panel of FIG. 2 shows a quantile regression plot; and the 15.87% percentile (−1 standard deviation), the 50.00% percentile (0 standard deviation) and the 84.13% percentile (+1 standard deviation) are plotted therein. The lower panel of FIG. 2 shows a final classification plot in which “high” refers to samples where the final standard score is greater than +2.0, “low” refers to samples where the final standard score is less than −2.0, and “Ok” refers to samples where the final standard score is greater than or equal to −2.0 and less than or equal to +2.0. Both the upper panel plots and the lower panel plots are based on samples from populations of most likely compliant subjects.

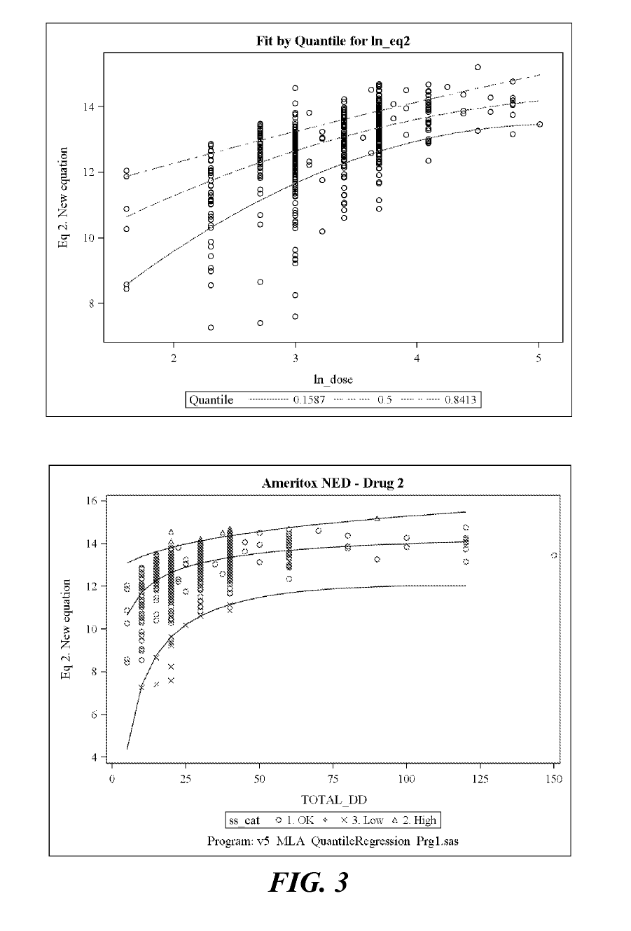

For oxycodone, the upper panel of FIG. 3 shows a quantile regression plot; and the 15.87% percentile (−1 standard deviation), the 50.00% percentile (0 standard deviation) and the 84.13% percentile (+1 standard deviation) are plotted therein. The lower panel of FIG. 3 shows a final classification plot for oxycodone in which “high” refers to samples where the final standard score is greater than +2.0, “low” refers to samples where the final standard score is less than −2.0, and “Ok” refers to samples where the final standard score is greater than or equal to −2.0 and less than or equal to +2.0. Both the upper panel plots and the lower panel plots are based on samples from populations of most likely compliant subjects.

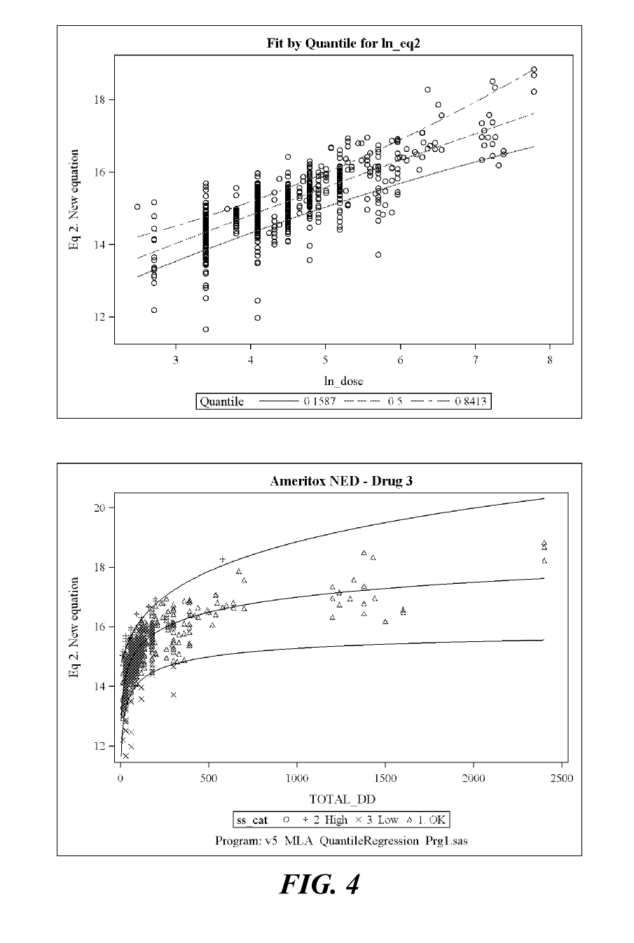

For controlled release morphine (MS CONTIN®) or morphine, the upper panel of FIG. 4 shows a quantile regression plot; and the 15.87% percentile (−1 standard deviation), the 50.00% percentile (0 standard deviation) and the 84.13% percentile (+1 standard deviation) are plotted therein. The lower panel of FIG. 4 shows a final classification plot for controlled release morphine (MS CONTIN®) or morphine in which “high” refers to samples where the final standard score is greater than +2.0, “low” refers to samples where the final standard score is less than −2.0, and “Ok” refers to samples where the final standard score is greater than or equal to −2.0 and less than or equal to +2.0. Both the upper panel plots and the lower panel plots are based on samples from populations of most likely compliant subjects.

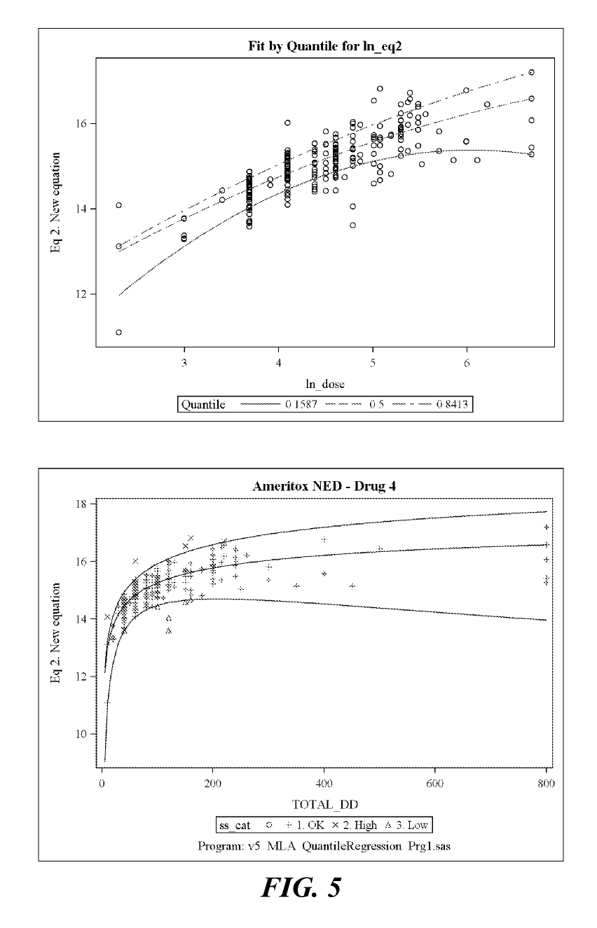

For extended release morphine (KADIAN®), the upper panel of FIG. 5 shows a quantile regression plot; and the 15.87% percentile (−1 standard deviation), the 50.00% percentile (0 standard deviation) and the 84.13% percentile (+1 standard deviation) are plotted therein. The lower panel of FIG. 5 shows a final classification plot for extended release morphine (KADIAN®)in which “high” refers to samples where the final standard score is greater than +2.0, “low” refers to samples where the final standard score is less than −2.0, and “Ok” refers to samples where the final standard score is greater than or equal to −2.0 and less than or equal to +2.0. Both the upper panel plots and the lower panel plots are based on samples from populations of most likely compliant subjects.

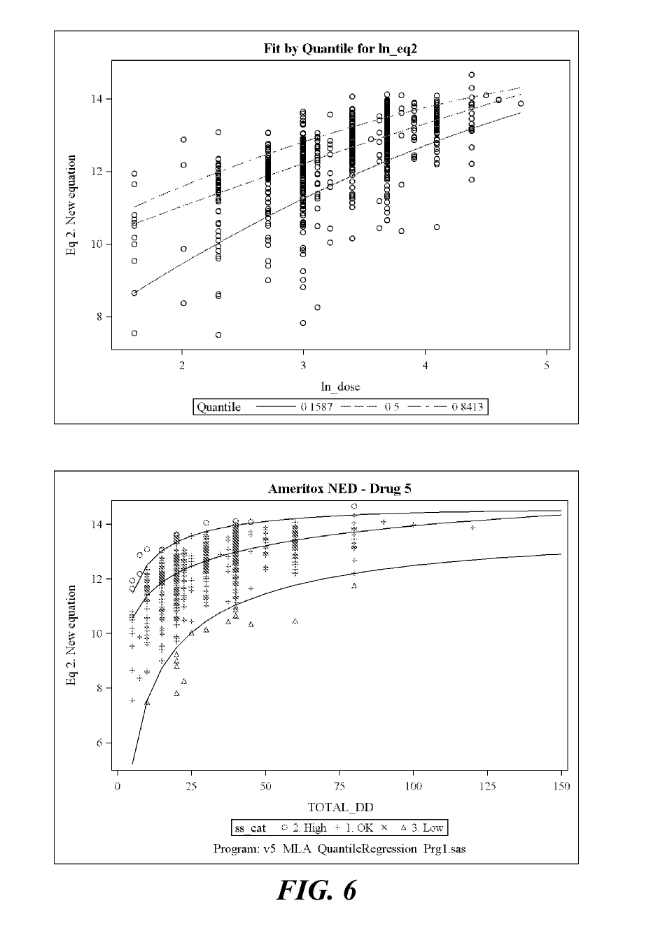

For hydrocodone, the upper panel of FIG. 6 shows a quantile regression plot; and the 15.87% percentile (−1 standard deviation), the 50.00% percentile (0 standard deviation) and the 84.13% percentile (+1 standard deviation) are plotted therein. The lower panel of FIG. 6 shows a final classification plot for hydrocodone in which “high” refers to samples where the final standard score is greater than +2.0, “low” refers to samples where the final standard score is less than −2.0, and “Ok” refers to samples where the final standard score is greater than or equal to −2.0 and less than or equal to +2.0. Both the upper panel plots and the lower panel plots are based on samples from populations of most likely compliant subjects.

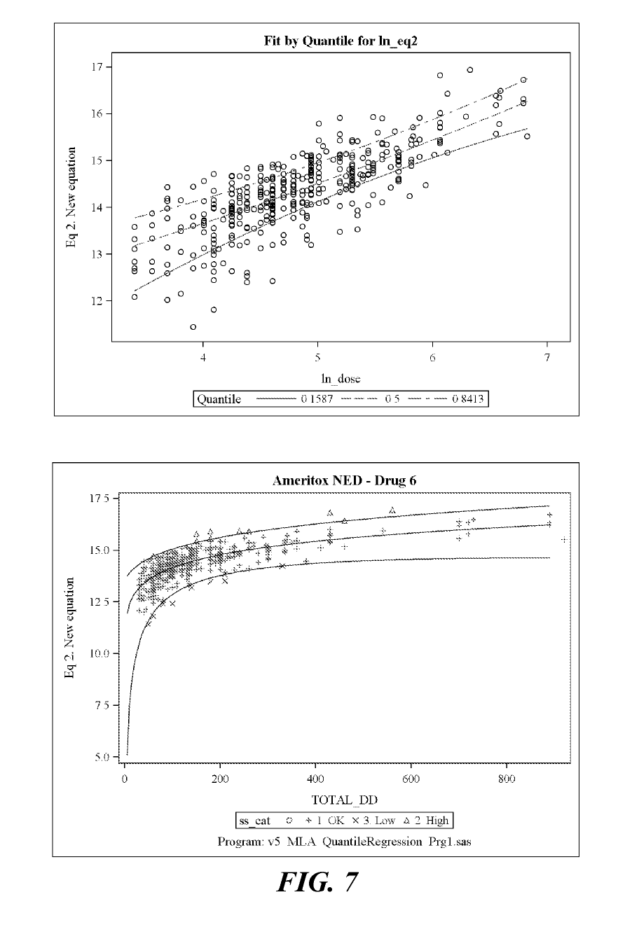

For the combination of controlled-release oxycodone (OXYCONTIN®) and oxycodone, the upper panel of FIG. 7 shows a quantile regression plot; and the 15.87% percentile (−1 standard deviation), the 50.00% percentile (0 standard deviation) and the 84.13% percentile (+1 standard deviation) are plotted therein. The lower panel of FIG. 7 shows a final classification plot for the combination of controlled-release oxycodone (OXYCONTIN®) and oxycodone in which “high” refers to samples where the final standard score is greater than +2.0, “low” refers to samples where the final standard score is less than −2.0, and “Ok” refers to samples where the final standard score is greater than or equal to −2.0 and less than or equal to +2.0. Both the upper panel plots and the lower panel plots are based on samples from populations of most likely compliant subjects.

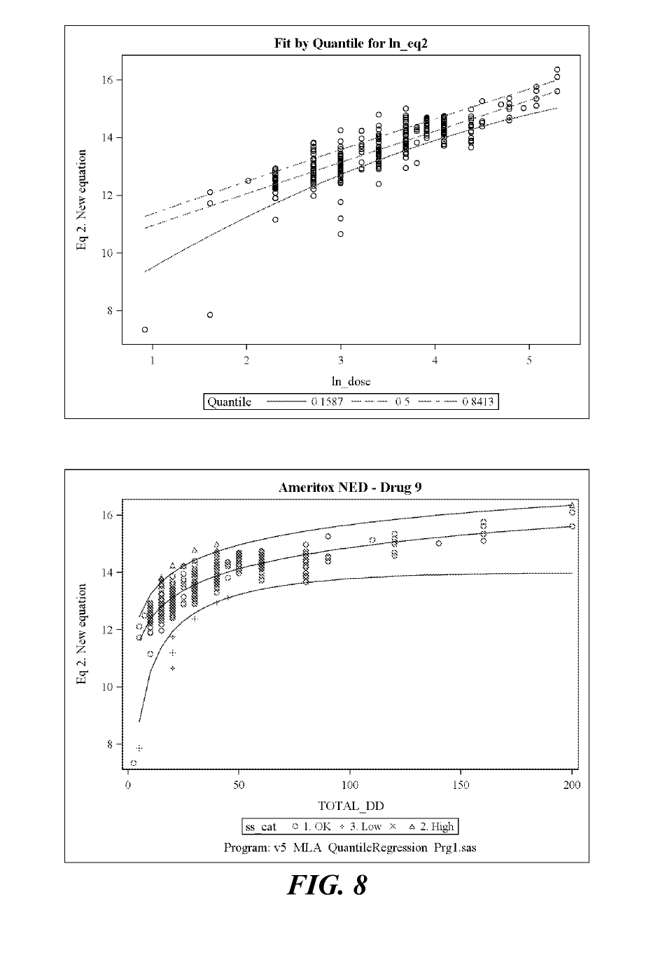

For methadone, the upper panel of FIG. 8 shows a quantile regression plot; and the 15.87% percentile (—1 standard deviation), the 50.00% percentile (0 standard deviation) and the 84.13% percentile (+1 standard deviation) are plotted therein. The lower panel of FIG. 8 shows a final classification plot for methadone in which “high” refers to samples where the final standard score is greater than +2.0, “low” refers to samples where the final standard score is less than −2.0, and “Ok” refers to samples where the final standard score is greater than or equal to −2.0 and less than or equal to +2.0. Both the upper panel plots and the lower panel plots are based on samples from populations of most likely compliant subjects.

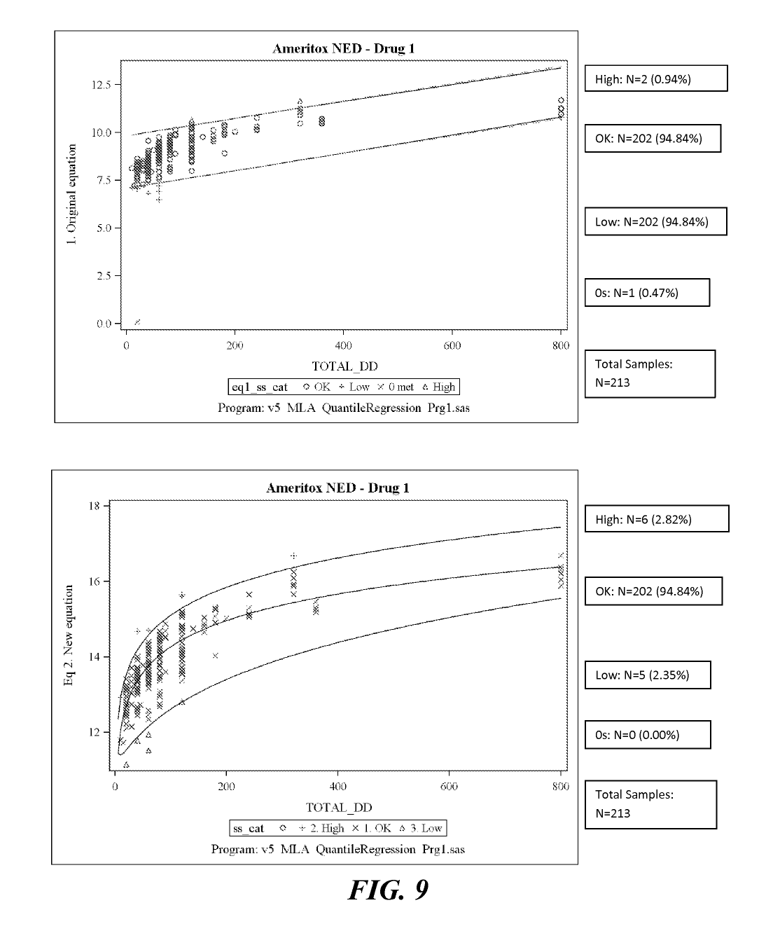

The lower panels of FIGS. 9-15 show plots of the results obtained from second order quantile regression analysis of normalized drug concentrations obtained from Equation 1. The upper panels of FIGS. 9-15 show plots of the results obtained from second order quantile regression analysis of the following normalization equation that is based solely on raw drug concentration, lean body weight and creatinine level:

$$ADJUSTED\_MET = ln ( \frac{METS * LBW}{UR\_CREAT} )$$

Lean body weight (LBW) refers to the difference between total body weight and body fat weight, and can be measured, calculated, or estimated using any suitable method or formula known to those skilled in the art.

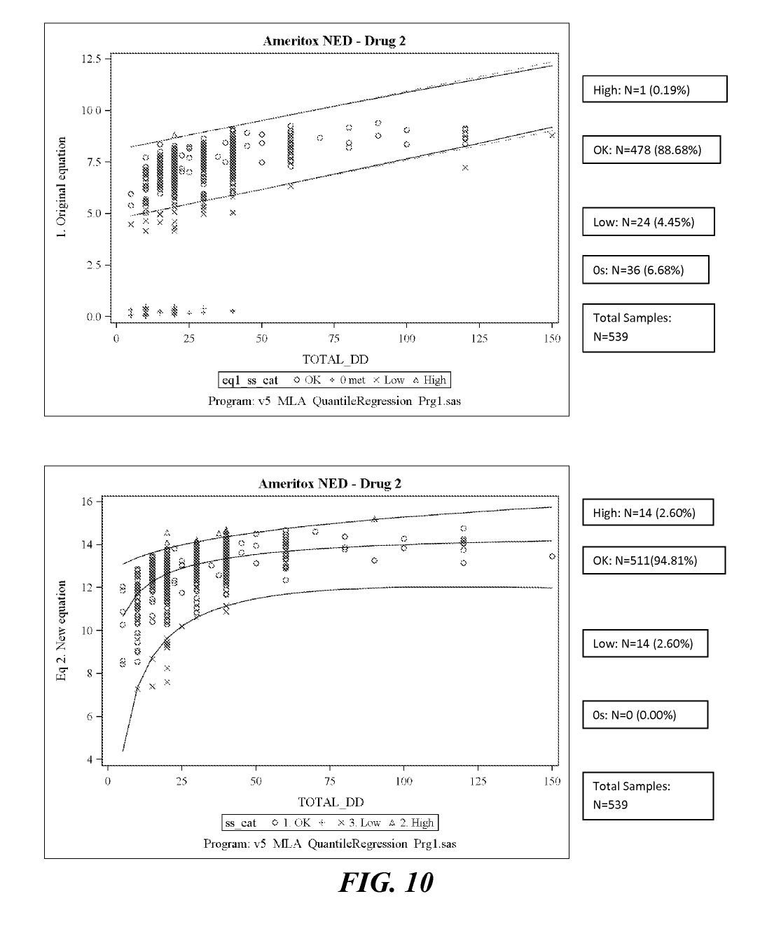

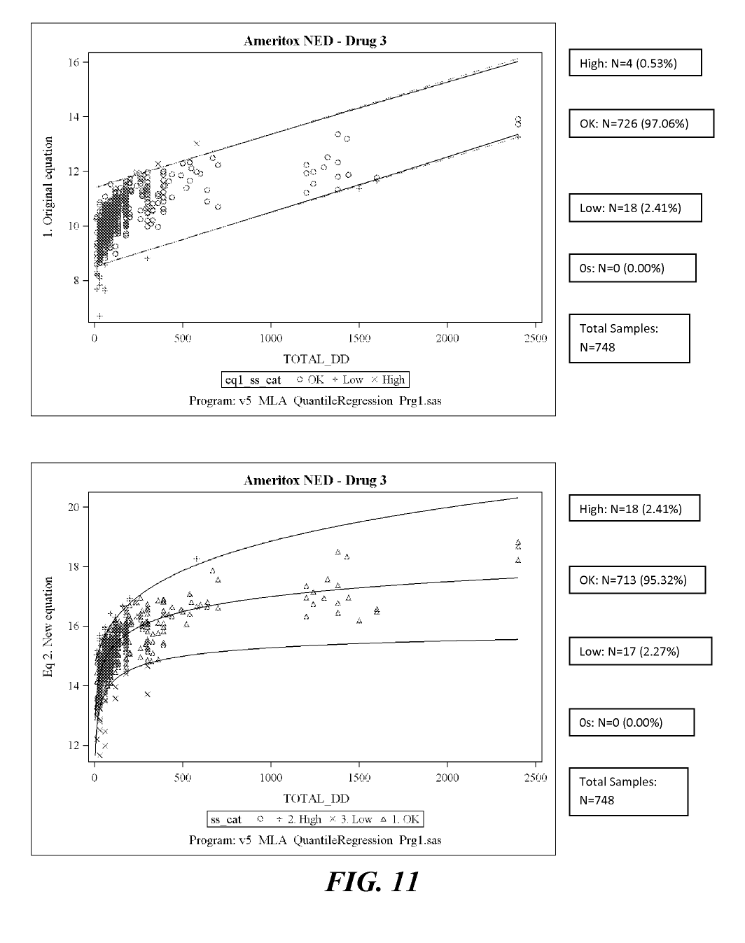

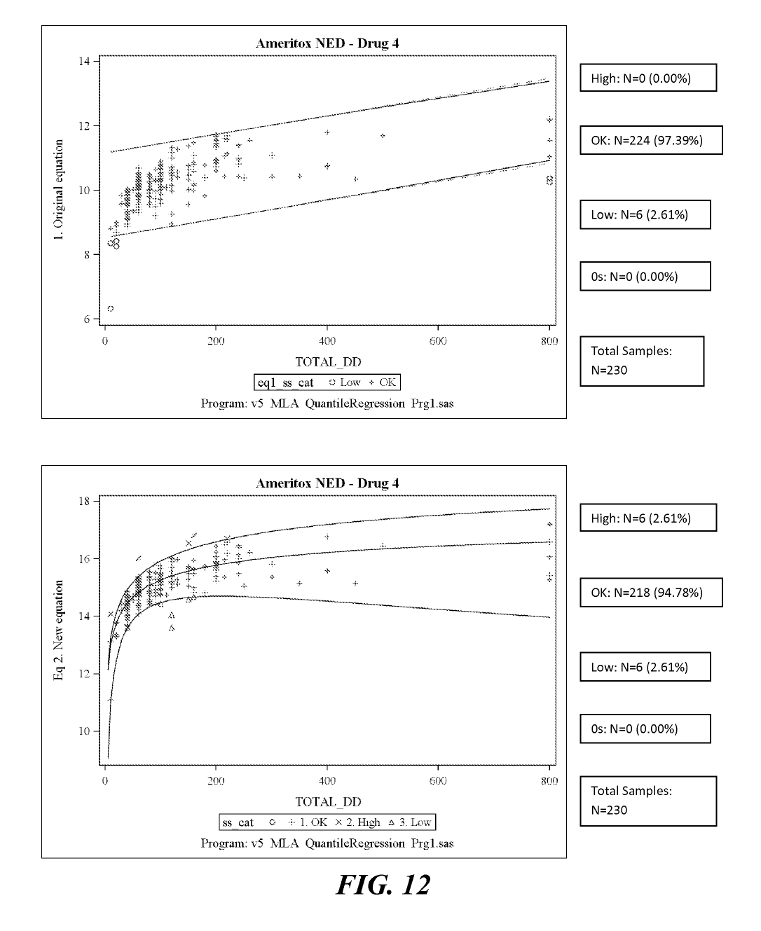

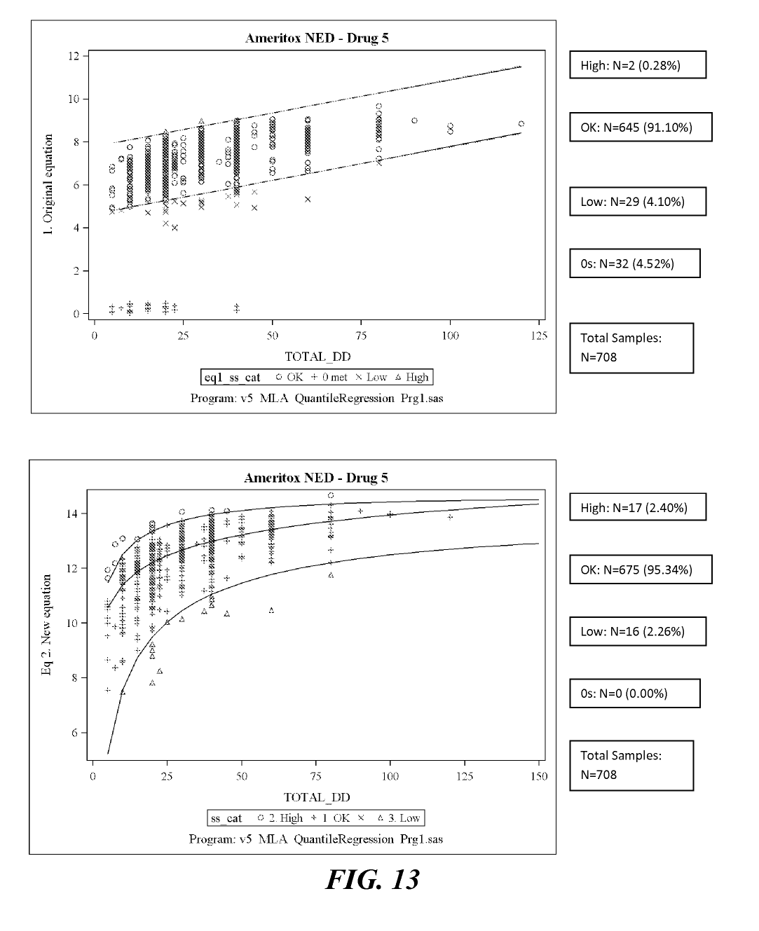

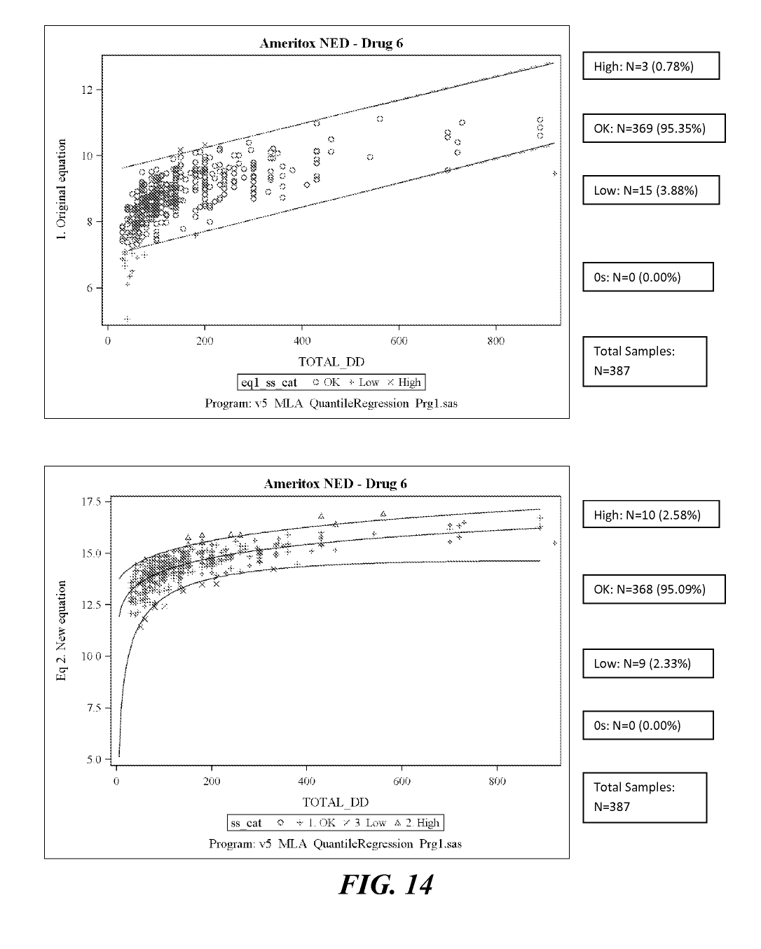

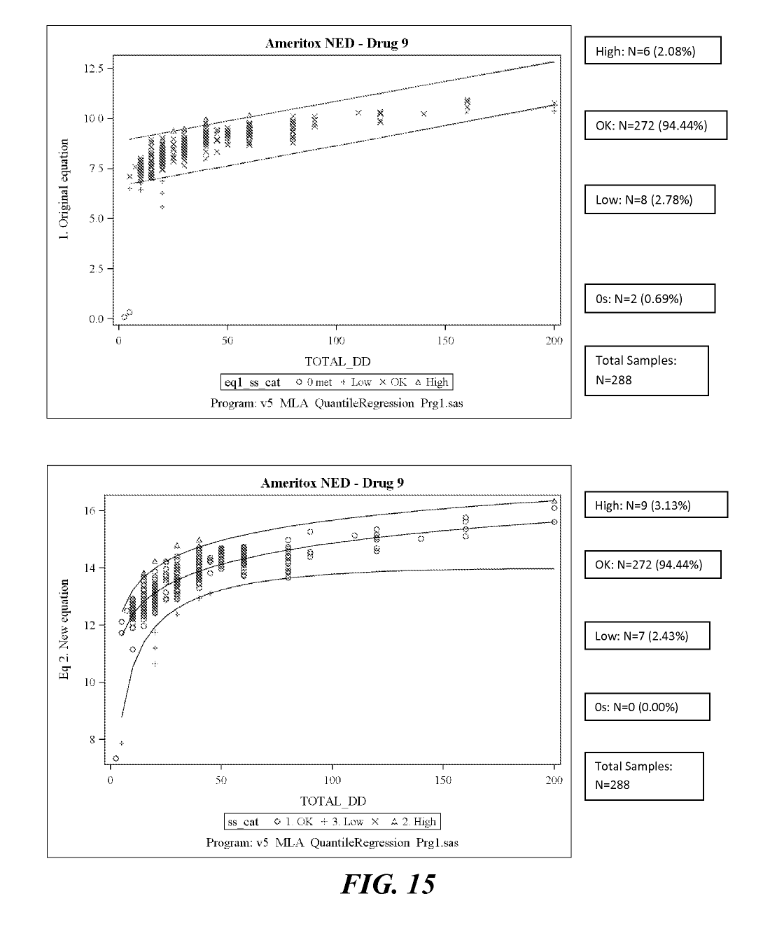

FIG. 9 shows corresponding data for controlled-release oxycodone (OXYCONTIN®), FIG. 10 shows corresponding data for oxycodone, FIG. 11 shows corresponding data for controlled release morphine (MS CONTIN®) or morphine, FIG. 12 shows corresponding data for extended release morphine (KADIAN®), FIG. 13 shows corresponding data for hydrocodone, FIG. 14 shows corresponding data for the combination of controlled-release oxycodone (OXYCONTIN®) and oxycodone, and FIG. 15 shows corresponding data for methadone.

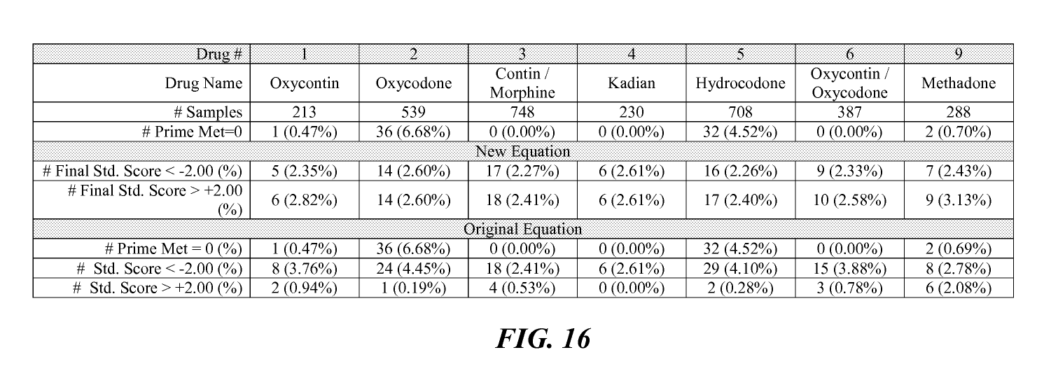

FIG. 16 shows a table in which the results obtained from second order quantile regression analysis of normalized drug concentrations obtained from Equation 1 are compared to the results obtained from second order quantile regression analysis of the above normalization equation that is based solely on raw drug concentration, lean body weight and creatinine level.

The method may be used in combination with any other method known to those skilled in the art for detecting a subject's potential non-compliance with a prescribed treatment protocol. Non-limiting examples of such methods include: interviews with the subject, fluid testing for the presence or absence of detectable levels of a drug, observation of the subject's behavior, appreciating reports of diversion of the subject's prescribed drug to others, etc. The method may be used in combination with any other method known to those skilled in the art for detecting a subject's potential non-compliance with a prescribed treatment protocol. Non-limiting examples of such methods include: interviews with the subject, fluid testing for the presence or absence of detectable levels of a drug, observation of the subject's behavior, appreciating reports of diversion of the subject's prescribed drug to others, etc.

In an embodiment, a method according to the present invention is used to reduce risk of drug misuse in a subject. In another embodiment, a method according to the present invention is used to confirm a subject's non-adherence to a chronic opioid therapy (COT) regimen. In yet another embodiment, a method according to the present invention provides a probability that a subject is non-compliant with a prescribed drug regimen. In an embodiment, a refined probability that the subject is non-compliant with a prescribed drug regimen results from the combination of the probability that the subject is non-compliant with a pretest probability.

In the above description, various methods have been described. It will be apparent to one of ordinary skill in the art that each of these methods may be implemented, in whole or in part, by software, hardware, and/or firmware. If implemented, in whole or in part, by software, the software may be stored on and executed by a tangible medium such as a CD-ROM, a floppy disk, a hard drive, a digital versatile disk (DVD), a read-only memory (ROM), etc.

### EXAMPLES

The following examples are for illustrative purposes only and are not to be construed as limiting the scope of the invention in any respect whatsoever.

### Example 1

### Controlled-Release Oxycodone (OXYCONTIN®)

A male subject with an age of 52 years, 168 days (52.46 years) and a weight of 220 lbs. is prescribed a 40 mg daily dose of controlled-release oxycodone (OXYCONTIN®). This prescribed daily dose is compared to the maximum dose for OXYCONTIN® of 800 mg/day from the table in FIG. 1, and the prescribed daily dose of the subject is less than the maximum daily dose.

Then fluid from the subject is tested. The urine concentration of the primary metabolite is 543 ng/ml, the urine concentration of the secondary metabolite is 324 ng/ml, and the urine creatinine level is 55.3 mg/dL.

Therefore, the normalized drug concentration is determined as follows:

$$\begin{matrix}{ADJUSTED}_{MET} = ln ( \frac{METS * NEW\_AGE * WEIGHT * NEW\_MF}{UR\_CREAT} ) \\ = ln ( \frac{( 543 + 324 ) * ( 140 - 52.46 ) * ( 220 ) * ( 1.0 )}{( 55.3 )} ) \\ = ln ( 301 \text{,} 941.76 ) \\ = 12.6180\end{matrix}$$

For the daily dose of 40 mg, the QR dose-based values can be determined using the LN_{DOSE} and the drug and dose-based coefficients in the table in FIG. 1 as follows:

$$\begin{matrix}L {N}_{DOSE} = ln ( daily dose ( mg ) ) \\ = ln ( 40 ) \\ = 3.6889 {QR}_{15.87} \\ = 10.9464 + ( 0.1547 * LN\_DOSE ) + ( 0.0909 * {LN\_DOSE}^{2} ) \\ = 10.9464 + ( 0.1547 * ( 3.6889 ) ) + ( 0.0909 * {( 3.6889 )}^{2} ) \\ = 12.7540 {QR}_{50.00} \\ = 9.9883 + ( 0.8703 * LN\_DOSE ) + ( 0.0130 * {LN\_DOSE}^{2} ) \\ = 9.9883 + ( 0.8703 * ( 3.6889 ) ) + ( 0.0130 * {( 3.6889 )}^{2} ) \\ = 13.3756 {QR}_{84.13} \\ = 10.5950 + ( 0.7687 * LN\_DOSE ) + ( 0.0268 * {LN\_DOSE}^{2} ) \\ = 10.5950 + ( 0.7687 * ( 3.6889 ) ) + ( 0.0268 * {( 3.6889 )}^{2} ) \\ = 13.7953\end{matrix}$$

Using the normalized drug concentration and the QR dose-based values, the preliminary standard score can be determined as follows:

ADJUSTED_{MET} (12.6180) is less than QR_{50.00} (13.3756), so

$${SS}_{Preliminary} = \frac{- ( {QR}_{50.00} - {ADJUSTED}_{MET} )}{( {QR}_{50.00} - {QR}_{15.87} )}$$

$${SS}_{Preliminary} = \frac{- ( 13.3756 - 12.6180 )}{( 13.3756 - 12.7540 )} = - 1.2188$$

Using the preliminary standard score with the adjustment variables obtained from the adjustment coefficients X₁ and X₂ from the table in FIG. 1, the final standard score can be determined as follows:

$${m}_{ss} = \frac{4.00}{{X}_{2} - {X}_{1}} = \frac{4.00}{( 1.94 - ( - 2.41 ) )} = 0.9195$$

$${b}_{ss} = - 2.00 - ( {m}_{ss} * {X}_{1} ) = - 2.00 - ( 0.9195 * ( - 2.41 ) ) = 0.2161$$

$$\begin{matrix}{SS}_{Final} = ( {m}_{ss} * {SS}_{Preliminary} ) + {b}_{ss} \\ = ( 0.9195 * ( - 1.2188 ) ) + 0.2161 \\ = - 0.9047\end{matrix}$$

Then the final standard score is evaluated by comparison to the upper threshold and the lower threshold. The final standard score of −0.9047 is greater than or equal to −2.00 and less than or equal to +2.00, so the subject may be classified as compliant as compared to the threshold.

### Example 2

### Oxycodone

A female subject with an age of 54 years, 33 days (54.09 years) and a weight of 136 lbs. is prescribed a 40 mg daily dose of oxycodone. This prescribed daily dose is compared to the maximum dose for oxycodone of 120 mg/day from the table in FIG. 1, and the prescribed daily dose of the subject is less than the maximum daily dose.

Then fluid from the subject is tested. The urine concentration of the primary metabolite is 0 ng/ml, the concentration of the secondary metabolite is 94 ng/ml, and the urine creatinine level is 15.7 mg/dL. The urine concentration of the primary metabolite is used as 15 ng/ml in the normalization equation instead of 0 ng/ml.

Therefore, the normalized drug concentration is determined as follows:

$$\begin{matrix}{ADJUSTED}_{MET} = ln ( \frac{METS * NEW\_AGE * WEIGHT * NEW\_MF}{UR\_CREAT} ) \\ = ln ( \frac{( 15 + 94 ) * ( 140 - 54.09 ) * ( 136 ) * ( 0.85 )}{( 15.7 )} ) \\ = ln ( 68 \text{,} 949.07 ) \\ = 11.1411\end{matrix}$$

The value of LN_{DOSE} can be determined as follows:

LN_{DOSE}=In(daily dose (mg))=In(40)=3.6889

LN_{DOSE} can be used with the drug and dose-based coefficients in the table in FIG. 1 to determine the QR dose-based values. The QR dose-based values can be used to determine a preliminary standard score for the normalized drug concentration of the subject. The preliminary standard score and the final standard score adjustment variables can be used to obtain the final standard score, and the final standard score can be compared to the lower threshold and the upper threshold to determine compliance.

### Example 3

### Controlled Release Morphine (MS CONTIN®) or Morphine

A male subject with an age of 65 years, 15 days (65.04 years) and a weight of 256.75 lbs. is prescribed a 60 mg daily dose of controlled release morphine (MS CONTIN®) or morphine. This prescribed daily dose is compared to the maximum dose for morphine/controlled release morphine (MS CONTIN®) of 599 mg/day from the table in FIG. 1, and the prescribed daily dose of the subject is less than the maximum daily dose.

Then fluid from the subject is tested. The urine concentration of the primary metabolite is 10,585 ng/ml, the urine concentration of the secondary metabolite is 73 ng/ml, and the urine creatinine level is 34.3 mg/dL. The urine concentration of the secondary metabolite of morphine is not used in the normalization equation.

Therefore, the normalized drug concentration is determined as follows:

$$\begin{matrix}{ADJUSTED}_{MET} = ln ( \frac{METS * NEW\_AGE * WEIGHT * NEW\_MF}{UR\_CREAT} ) \\ = ln ( \frac{( 10585 ) * ( 140 - 65.04 ) * ( 256.75 ) * ( 1.0 )}{( 34.3 )} ) \\ = ln ( 5 \text{,} 939 \text{,} 320.65 ) \\ = 15.5971\end{matrix}$$

The value of LN_{DOSE} can be determined as follows:

LN_{DOSE}=In(daily dose (mg))=In(60)=4.0943

LN_{DOSE} can be used with the drug and dose-based coefficients in the table in FIG. 1 to determine the QR dose-based values. The QR dose-based values can be used to determine a preliminary standard score for the normalized drug concentration of the subject. The preliminary standard score and the final standard score adjustment variables can be used to obtain the final standard score, and the final standard score can be compared to the lower threshold and the upper threshold to classify a subject as compliant.

### Example 4

### Extended Release Morphine (KADIAN®)

A female subject with an age of 51 years, 99 days (51.27 years) and a weight of 288.75 lbs. is prescribed a 60 mg daily dose of extended release morphine (KADIAN®). This prescribed daily dose is compared to the maximum dose for extended release morphine (KADIAN®) of 214 mg/day from the table in FIG. 1, and the prescribed daily dose of the subject is less than the maximum daily dose.

Then fluid from the subject is tested. The urine concentration of the primary metabolite is 22,994 ng/ml, the urine concentration of the secondary metabolite is 0 ng/ml, and the urine creatinine level is 55.3 mg/dL. The urine concentration of the secondary metabolite of extended release morphine (KADIAN®) is not used in the normalization equation.

Therefore, the normalized drug concentration is determined as follows:

$$\begin{matrix}{ADJUSTED}_{MET} = ln ( \frac{METS * NEW\_AGE * WEIGHT * NEW\_MF}{UR\_CREAT} ) \\ = ln ( \frac{( 22994 ) * ( 140 - 51.27 ) * ( 288.75 ) * ( 0.85 )}{( 55.3 )} ) \\ = ln ( 9 \text{,} 055 \text{,} 257.32 ) \\ = 16.0189\end{matrix}$$

The value of LN_{DOSE} can be determined as follows:

LN_{DOSE}=In(daily dose (mg))=In(60)=4.0943

LN_{DOSE} can be used with the drug and dose-based coefficients in the table in FIG. 1 to determine the QR dose-based values. The QR dose-based values can be used to determine a preliminary standard score for the normalized drug concentration of the subject. The preliminary standard score and the final standard score adjustment variables can be used to obtain the final standard score, and the final standard score can be compared to the lower threshold and the upper threshold to help determine compliance.

### Example 5

### Hydrocodone

A male subject with an age of 31 years, 37 days (31.1 years) and a weight of 184 lbs. is prescribed a 15 mg daily dose of hydrocodone. This prescribed daily dose is compared to the maximum dose for hydrocodone of 80 mg/day from the table in FIG. 1, and the prescribed daily dose of the subject is less than the maximum daily dose.

Then fluid from the subject is tested. The urine concentration of the primary metabolite is 720 ng/ml, the concentration of the secondary metabolite is 239 ng/ml, and the urine creatinine level is 121.5 mg/dL.

Therefore, the normalized drug concentration is determined as follows:

$$\begin{matrix}{ADJUSTED}_{MET} = ln ( \frac{METS * NEW\_AGE * WEIGHT * NEW\_MF}{UR\_CREAT} ) \\ = ln ( \frac{( 720 + 239 ) * ( 140 - 31.1 ) * ( 184 ) * ( 1.0 )}{( 121.5 )} ) \\ = ln ( 158 \text{,} 156.86 ) \\ = 11.9713\end{matrix}$$

The value of LN_{DOSE} can be determined as follows:

LN_{DOSE}=In(daily dose (mg))=In(15)=2.7081

LN_{DOSE} can be used with the drug and dose-based coefficients in the table in FIG. 1 to determine the QR dose-based values. The QR dose-based values can be used to determine a preliminary standard score for the normalized drug concentration of the subject. The preliminary standard score and the final standard score adjustment variables can be used to obtain the final standard score, and the final standard score can be compared to the lower threshold and the upper threshold to help determine compliance.

### Example 6

### Controlled-Release Oxycodone (OXYCONTIN®) and Oxycodone

A female subject with an age of 29 years, 329 days (29.9 years) and a weight of 160.5 lbs. is prescribed a 100 mg daily dose of the combination of controlled-release oxycodone (OXYCONTIN®) and oxycodone. This prescribed daily dose is compared to the maximum dose for the combination of controlled-release oxycodone (OXYCONTIN®) and oxycodone of 299 mg/day from the table in FIG. 1, and the prescribed daily dose of the subject is less than the maximum daily dose.

Then fluid from the subject is tested. The urine concentration of the primary metabolite is 4,070 ng/ml, the concentration of the secondary metabolite is 0 ng/ml, and the urine creatinine level is 246.4 mg/dL. The urine concentration of the secondary metabolite is used as 15 ng/ml in the normalization equation instead of 0 ng/ml.

Therefore, the normalized drug concentration is determined as follows:

$$\begin{matrix}{ADJUSTED}_{MET} = ln ( \frac{METS * NEW\_AGE * WEIGHT * NEW\_MF}{UR\_CREAT} ) \\ = ln ( \frac{( 4070 + 15 ) * ( 140 - 29.9 ) * ( 160.5 ) * ( 0.85 )}{( 246.4 )} ) \\ = ln ( 249 \text{,} 019.09 ) \\ = 12.4253\end{matrix}$$

The value of LN_{DOSE} can be determined as follows:

LN_{DOSE}=In(daily dose (mg))=In(100)=4.6052

LN_{DOSE} can be used with the drug and dose-based coefficients in the table in FIG. 1 to determine the QR dose-based values. The QR dose-based values can be used to determine a preliminary standard score for the normalized drug concentration of the subject. The preliminary standard score and the final standard score adjustment variables can be used to obtain the final standard score, and the final standard score can be compared to the lower threshold and the upper threshold to help determine compliance.

### Example 7

### Methadone

A male subject with an age of 39 years, 197 days (39.54 years) and a weight of 167 lbs. is prescribed a 40 mg daily dose of methadone. This prescribed daily dose is compared to the maximum dose for methadone of 79 mg/day from the table in FIG. 1, and the prescribed daily dose of the subject is less than the maximum daily dose.

Then fluid from the subject is tested. The urine concentration of the primary metabolite is 5,958 ng/ml, and the urine creatinine level is 104.8 mg/dL.

Therefore, the normalized drug concentration is determined as follows:

$$\begin{matrix}{ADJUSTED}_{MET} = ln ( \frac{METS * NEW\_AGE * WEIGHT * NEW\_MF}{UR\_CREAT} ) \\ = ln ( \frac{( 5958 ) * ( 140 - 39.54 ) * ( 167 ) * ( 1.0 )}{( 104.8 )} ) \\ = ln ( 953 \text{,} 781.78 ) \\ = 13.7682\end{matrix}$$

The value of LN_{DOSE} can be determined as follows:

LN_{DOSE}=In(daily dose (mg))=In(40)=3.6889

LN_{DOSE} can be used with the drug and dose-based coefficients in the table in FIG. 1 to determine the QR dose-based values. The QR dose-based values can be used to determine a preliminary standard score for the normalized drug concentration of the subject. The preliminary standard score and the final standard score adjustment variables can be used to obtain the final standard score, and the final standard score can be compared to the lower threshold and the upper threshold to help determine compliance.

I/We claim:

1. A method of determining non-compliance with a prescribed drug regiment in a subject, the method comprising:
determining a prescribed daily dose of drug in a subject;
determining an age, a weight and a gender of the subject;
measuring a concentration of creatinine and a concentration of a primary metabolite of the drug in urine of the subject;
determining a normalized metabolite concentration as a function of parameters comprising the concentration of creatinine, the concentration of the primary metabolite, the age, the weight and the gender of the subject;
determining a quantile regression dose-based value for each of a plurality of standard deviation values, the plurality of standard deviation values relative to a mean normalized drug concentration derived from samples of a population;
comparing the normalized metabolite concentration to the quantile regression dose-based value for one of the plurality of standard deviation values;
determining a preliminary standard score based at least partially on comparison of the normalized metabolite concentration to the quantile regression dose-based value;
determining a final standard score based at least partially on the preliminary standard score; and
comparing the final standard score to an upper threshold and a lower threshold that are predetermined and standardized.

2. The method of claim 1 further comprising measuring a concentration of a secondary metabolite in the urine of the subject, wherein the parameters used in determining the normalized metabolite concentration comprise the concentration of the secondary metabolite.

3. The method of claim 2 wherein the parameters used in determining the normalized metabolite concentration consist of the concentration of creatinine, the concentration of the primary metabolite, the concentration of the secondary metabolite, the age, the weight and the gender of the subject.

4. The method of claim 1 wherein the plurality of standard deviation values are a −1 standard deviation value, a 0 standard deviation value, and a +1 standard deviation value.

5. The method of claim 4 further comprising comparing the normalized metabolite concentration to the quantile regression dose-based value for the 0 standard deviation value.

6. The method of claim 5 further comprising comparing the normalized metabolite concentration to at least one of the quantile regression dose-based value for the −1 standard deviation value or the quantile regression dose-based value for the +1 standard deviation value.

7. The method of claim 1 further comprising determining if the prescribed daily dose of the drug is less than a maximum daily dose of the drug before measuring the concentration of creatinine and the concentration of the primary metabolite of the opioid in the urine.

8. The method of claim 1 wherein the quantile regression dose-based value is based at least partially on a natural log of the prescribed daily dose.

9. The method of claim 1 wherein the quantile regression dose-based value is based at least partially on one or more coefficients derived from the samples of the population.

10. The method of claim 1 wherein the final standard score is based at least partially on one or more adjustment variables derived from the samples of the population.

11. The method of claim 1 wherein a plurality of members are assigned to the population based on a dose administered to the plurality of members and the presence or absence of one or more exclusion criteria selected from the group consisting of high drug metabolism, low drug metabolism, lab abnormalities, impaired kidney or liver function, use of drugs with overlapping metabolites on the same day, an inconsistent schedule of medication administration, and combinations thereof.

12. The method of claim 1 wherein the drug is selected from the group consisting of controlled-release oxycodone, oxycodone, controlled release morphine, morphine, extended release morphine hydrocodone, methadone, and a combination of controlled-release oxycodone and oxycodone.

13. The method of claim 1 wherein the parameters consist of the concentration of creatinine, the concentration of the primary metabolite, the age, the weight and the gender of the subject.

14. The method of claim 1 further comprising determining if the subject is compliant with a drug regimen that includes the prescribed daily dose of the drug.

15. The method of claim 1 wherein the primary metabolite is the drug.

16. The method of claim 1 wherein the drug is an opioid or an antipsychotic drug.
